(19) United States
(12) Patent Application Publication
(10) Pub. No.: US 2016/0304415 A1
(43) Pub. Date: Oct. 20, 2016

(54) WORK-UP OF A REACTION MIXTURE (RM) COMPRISING CYCLODODECATRIENE AND AN ACTIVE CATALYST SYSTEM

(71) Applicant: BASF SE, Ludwigshafen (DE)

(72) Inventors: Michael Schwartztrauber, Neustadt (DE); Michael SCHELPER, Weinheim (DE); Hugo Rafael GARCIA ANDARCIA, Shanghai (CN); Avni TURKSEVEN, Schifferstadt (DE); Rocco PACIELLO, Bad Dürkheim (DE); THOMAS SCHAUB, Neustadt (DE)

(21) Appl. No.: 15/102,659

(22) PCT Filed: Dec. 9, 2014
(86) PCT No.: PCT/EP2014/077012

§ 371 Date: Jun. 8, 2016

(30) Foreign Application Priority Data
Dec. 11, 2013 (EP) 13196667.3

(51) Int. Cl. C07C 7/00; B01J 31/38; B01J 31/14; B01J 31/22; C07C 7/04; C07C 7/148

(52) U.S. Cl. CPC C07C 7/005; C07C 7/04; C07C 7/14858; C07C 7/14866; B01J 31/143; B01J 31/2234; B01J 31/38; C07C 2101/20; C07C 2531/14; C07C 2531/38; C07C 2531/22; B01J 2231/20; B01J 2531/46

(57) ABSTRACT

The application relates to a process for the work-up of a reaction mixture (R_{M}) comprising cyclododecatriene and an active catalyst system (C) comprising an organoaluminum compound, said process comprising the steps of:
a) contacting the reaction mixture (R_{M}) with gaseous ammonia to obtain a first mixture (M1),
b) contacting the first mixture (M1) with water to obtain a second mixture (M2),
c) distillatively removing cyclododecatriene from the second mixture (M2).

The present invention relates to a process for the work-up of a reaction mixture (R_{M}) comprising cyclododecatriene and an active catalyst system.

Cyclododecatriene is abbreviated to CDT hereinbelow. The quality of cyclododecanone and laurolactam which are descendant products obtainable from CDT depends decisively on the purity of the CDT starting material used. Care must therefore be taken at the CDT stage to very substantially remove compounds such as vinylcyclohexene, cyclooctadiene. C₁₆ compounds, oligomers and polymer compounds. In practice, a CDT purity of more than 99% is generally required to achieve a sufficient product quality of the descendant products cyclododecanone and laurolactam. CDT is produced by the trimerization of butadiene. By-products of CDT production include vinylcyclohexene, cyclooctadiene and oligomers and polymers of butadiene having 16, 20 or more carbon atoms. Depending on the catalyst used. CDT production may moreover also generate mono- and polychlorinated analogues of virtually all of the components described hereinabove. The cyclotrimerization of butadiene to give cyclododecatriene using Ziegler catalyst systems is of industrial importance since it provides a route to the cycloaliphatic and open-chain C₁₂ compound classes. CDT descendant products of interest include, inter alia, cyclododecanone, cyclododecanol, decanedicarboxylic acid and laurolactam. Laurolactam is of particular interest since it is an intermediate in the production of nylon 12.

The trimerization of butadiene to give cyclododecatriene (CDT) is generally carried out under transition metal catalysis using, for example, titanium, nickel or chromium catalysts reduced with a reducing agent. The reducing agent is generally an organometallic compound from the first to third main groups of the periodic table and organoaluminum compounds, for example aluminum alkyls, have turned out to be advantageous. The use of titanium-based catalysts such as titanium tetrachloride and titanium acetylacetonate in combination with aluminum alkyls has proven particularly advantageous in industry.

Useful catalyst systems are described in U.S. Pat. No. 3,878,258, U.S. Pat. No. 3,655,795 and DE 3 021 840 for example. The catalyst systems employed preferably comprise titanium tetrachloride and also ethylaluminum sesquichloride or diethylaluminum chloride. Here, the ratio of Al:Ti is generally 4:1 or higher in order to minimize the formation of polybutadiene.

Typical reaction temperatures of such Ziegler catalysts are generally in the range of from 40° C. to 90° C. Titanium catalysts give mainly the cis,trans,trans isomer of 1,5,9-cyclododecatriene. Nickel or chromium catalysts give mainly the all-trans isomer of 1,5,9-cyclododecatriene. The trimerization reaction of butadiene can be carried out in the presence of an apolar inert solvent, for example benzene, cyclohexane, hexane, heptane, octane, decane, toluene or xylene. Trimerization can moreover also be carried out without adding a solvent. Such reactions are described in printed publications DE 3 021 840 and U.S. Pat. No. 6,403,851 for example.

The reactivity and selectivity of the catalyst system can be modified and improved by addition of one or more promoters. Useful promoters are described in printed publications U.S. Pat. No. 3,546,309, DE 2 825 341 and U.S. Pat. No. 3,381,045 for example.

The trimerization of butadiene to give cyclododecatriene thus generally gives a reaction mixture comprising the active catalyst systems described hereinabove as well as cyclododecatriene and any further by-products. To further work up the cyclododecatriene target product, the active catalyst system needs to be either removed or deactivated. Further work-up of the reaction mixture is generally effected by distillative removal of the cyclododecatriene. Distillative removal of the cyclododecatriene in the presence of the active catalyst would lead to the formation of by-products on a massive scale and to the destruction of some or all of the cyclododecatriene.

DE 3 321 840 describes a process wherein the active catalyst system is employed in the form of a supported catalyst on polystyrene. The supported catalyst is removed prior to the distillative removal of the cyclododecatriene. However, this process is costly and inconvenient as it necessarily comprises an additional removal step.

Homogeneously catalyzed trimerization reactions of butadiene are therefore generally preferred. US patent documents U.S. Pat. No. 3,655,795 and U.S. Pat. No. 3,878,259 employ gaseous ammonia to deactivate the active catalyst system. Here, the reaction mixture is saturated with gaseous ammonia.

Gaseous ammonia deactivates the active catalyst system, thereby preventing formation of by-products and destruction of the cyclododecatriene during the distillative removal of the cyclododecatriene. However, deactivation with gaseous ammonia generates vaporizable aluminum compounds from the organoaluminum compounds, for example ethylaluminum sesquichloride (Al₂Cl₃(C₂H₅)₃), comprised in the active catalyst system. Deactivation with gaseous ammonia may moreover generate precipitates which need to be filtered off prior to the distillative work-up.

It is believed that reaction of ammonia with the organoaluminum compounds forms amidoaluminum chlorides of empirical formulae AlCl₂NH₂ or AlCl₃NH₃. Relevant research is described, for example, in J. Chem. Soc. 1965, pp. 1092 to 1096 and J. Am. Chem. Soc. 1960, 83, pp. 542 to 546. These amidoaluminum chlorides are vaporized during the distillative removal of cyclododecatriene and thus cause problems in the work-up of the cyclododecatriene since they form deposits in heat exchangers for example and such deposits then require a very great deal of cleaning to remove. It is believed that once vaporized and/or during vaporization the amidoaluminum chlorides undergo a condensation reaction with one another to form polymeric condensation products responsible for the deposits. The authors of J. Chem. Soc. 1965, pp. 1092 to 1096 and J. Am. Chem. Soc. 1960, 83, pp. 542 to 546 believe that the condensation products (deposits) have the empirical formulae (AlN)ₓ and/or (AlClNH)ₓ. In addition, the presence of water in later work-up steps (e.g. washing the crude mixture following the vaporization) can bring about the hydrolysis of these amidoaluminum chlorides to form insoluble aluminum oxide. This too can lead to undesired deposits which necessitate additional plant cleaning.

U.S. Pat. No. 3,381,045 and U.S. Pat. No. 3,546,309 describe the use of isopropyl alcohol and/or acetone to deactivate the active catalyst system. This avoids the formation of vaporizable amidoaluminum chlorides and the formation of deposits associated therewith. However, the use of polar solvents such as alcohols generates two liquid phases which need to be separated prior to the distillative work-up.

The use of polar solvents such as isopropyl alcohol or acetone additionally brings about the formation of precipitates and also rag formation. Rag formation impedes further work-up of the cyclododecatriene. The precipitates need to be filtered off. Rag formation additionally impedes phase separation of the two liquid phases. These deactivation methods lead to the formation of a third phase of rag, also known as sludge, between the two liquid phases, said rag comprising both liquid and solids. Work-up of the reaction mixture after deactivation of the active catalyst system is therefore extremely difficult to realize on a large industrial scale using the processes described in U.S. Pat. No. 3,381,045 and U.S. Pat. No. 3,546,309. The use of polar solvents such as alcohols or acetone to deactivate the active catalyst system has the additional disadvantage that said solvents favor the formation of chlorinated by-products and this negatively affects the product quality of the cyclododecatriene and makes it harder to obtain on-spec cyclododecatriene.

DE 1 768 067 describes a process for the work-up of reaction mixtures comprising cyclododecatriene wherein a concentrated aqueous ammonia solution is used to deactivate the active catalyst system. Addition of the concentrated aqueous ammonia solution is preferably followed by further addition of water or a 20% strength aqueous sodium hydroxide solution. The DE 1 768 067 work-up procedure too generates a precipitate when the concentrated aqueous ammonia solution is added and said precipitate needs to be filtered off. The preferred addition of water further favors the formation of the precipitate. When addition of the concentrated aqueous ammonia solution is followed by addition of an aqueous sodium hydroxide solution, the precipitate is redissolved but two liquid phases requiring separation are formed in any event. It is thus mandatory also in the DE 1 768 067 process that the precipitate formed be filtered off or that the two liquid phases formed be subjected to a phase separation.

The processes described in the prior art for work-up of reaction mixtures comprising cyclododecatriene and an active catalyst system are therefore difficult to realize on a large industrial scale. This is because it is mandatory in these processes that the precipitate formed be filtered off or that the two liquid phases formed be subjected to a phase separation. The processes described in the prior art which employ gaseous ammonia to deactivate the active catalyst system have the additional disadvantage that in the subsequent distillative removal of cyclododecatriene, vaporizable amidoalumium chlorides lead to deposits which impede continuous distillative removal of cyclododecatriene.

It is thus an object of the present invention to provide a process which does not exhibit the disadvantages of the prior art or which exhibits them only to a lesser extent. The process shall in particular provide a work-up for a reaction mixture comprising cyclododecatriene and an active catalyst system wherein a phase separation of two liquid phases is not necessary. Filtering off precipitates shall moreover be very substantially avoided. The process shall moreover prevent the formation of deposits generated in the distillative work-up of cyclododecatriene by vaporizable amidoaluminum chlorides. The process according to the invention shall additionally be inexpensive and economical and shall comprise fewer process steps than the processes described in the prior art.

This object is achieved by a process for the work-up of a reaction mixture (R_{M}) comprising cyclododecatriene and an active catalyst system (C) comprising an organoaluminum compound, said process comprising the steps of:
a) contacting the reaction mixture (R_{M}) with gaseous ammonia to obtain a first mixture (M1),
b) contacting the first mixture (M1) with water to obtain a second mixture (M2),
c) distillatively removing cyclododecatriene from the second mixture (M2).

It was found that, surprisingly, the process according to the invention provides an improved and in particular more economical work-up of reaction mixtures (R_{M}) comprising cyclododecatriene and an active catalyst system (C) comprising an organoaluminum compound. The process according to the invention need not comprise a phase separation following deactivation of the active catalyst system (C) since the second mixture (M2) obtained in process step b) preferably comprises only one liquid phase. The process according to the invention has the additional advantage that the contacting of the first mixture (M1) with water according to process step b) safely destroys vaporizable amidoaluminum chlorides, thereby preventing the formation of deposits in the distillative removal of the cyclododecatriene according to process step c). The process according to the invention moreover generates very little, if any, precipitate which needs to be filtered off. The filters employed accordingly require only infrequent replacement which favors running the process according to the invention as a continuous operation. Surprisingly, a large part of the second deactivated catalyst system (C2) remains in the liquid phase on completion of the process according to the invention and may be disposed of easily as bottoms from the distillation apparatus following the distillative removal of the cyclododecatriene.

### Reaction Mixture (R_{M})

The reaction mixture (R_{M}) comprises cyclododecatriene and an active catalyst system (C) comprising an organoaluminum compound. The reaction mixture (R_{M}) generally derives from a homogeneously catalyzed butadiene trimerization reaction.

In accordance with the invention, cyclododecatriene is understood to mean all isomers of cyclododecatriene. Cyclododecatriene, more precisely 1,5,9-cyclododecatriene, can exist as four different isomers.

These are Z,Z,Z-1,5,9-cyclododecatriene (CAS No. 4736-48-5), E,E,E-1,5,9-cyclododecatriene (CAS No. 676-22-2), E,Z,Z-1,5,9-cyclododecatriene (CAS No. 2765-29-9) and E,E,Z-1,5,9-cyclododecatriene (CAS No. 706-31-0).

The trimerization (cyclotrimerization) of 1,3-butadiene to give cyclododecatriene generally gives mixtures of the abovementioned isomers. The ratio of the isomers to one another may be controlled by the choice of active catalyst system (C) employed. Thus, for example, catalyst systems (C) comprising nickel or chromium give predominantly Z,Z,Z-1,5,9-cyclododecatriene, whereas active catalyst systems (C) comprising titanium give mainly E,Z,Z-1,5,9-cyclododecatriene. This is also known as 1,5,9-trans-trans-cis-cyclododecatriene. The type of isomers comprised in the reaction mixture (R_{M}) does not constitute an essential feature of the invention.

Nevertheless, the reaction mixture (R_{M}) preferably comprises mainly E,E,Z-1,5,9-cyclododecatriene. In one particularly preferred embodiment, the reaction mixture (R_{M}) comprises at least 80% by weight, preferably at least 90% by weight, of E,E,Z-1,5,9-cyclododecatriene based on the total weight of all cyclododecatriene isomers comprised in the reaction mixture (R_{M}).

The reaction mixture (R_{M}) preferably derives from a trimerization reaction of 1,3-butadiene in the presence of an active catalyst system (C) comprising an organoaluminum compound and a titanium compound of oxidation state +IV. It is particularly preferred when the reaction mixture (R_{M}) comprises an active catalyst system (C) obtainable from at least one organoaluminum compound selected from the group consisting of Al₂(C₂H₅)₆, Al₂Cl₃(C₂H₅)₃ and AlCl(C₂H₅)₂ and at least one titanium compound selected from the group consisting of titanium acetylacetonate and titanium chloride. It is especially preferred when the reaction mixture (R_{M}) comprises an active catalyst system formed from titanium tetrachloride (TiCl₄) and ethylaluminum sesquichloride Al₂Cl₃(C₂H₅)₃.

The trimerization of 1,3-butadiene to give 1,5,9-cyclododecatriene is generally carried out using 0.0001 to 0.1 mol of an organoaluminum compound, 0.00001 to 0.01 mol of a titanium compound of oxidation state +IV and optionally 0.00001 to 0.01 mol of a promoter per 1 mol of 1,3-butadiene.

When a promoter is used it is particularly preferable when said promoter is water.

The active catalyst system (C) is preferably generated using 2 to 50 mol of the organoaluminum compound and 0.1 to 20 mol of the promoter, preferably water, per 1 mol of the titanium compound of oxidation state +IV.

The trimerization of 1,3-butadiene can preferably be carried out in the presence of an apolar solvent. The trimerization may moreover also be carried out in the absence of such an apolar solvent. Particularly preferred apolar solvents are solvents inert under the reaction conditions employed in the trimerization of 1,3-butadiene. In accordance with the invention, “inert” is understood to mean that the inert apolar solvents remain chemically unchanged under the reaction conditions employed in the trimerization of 1,3-butadiene.

Particular preference is given to a reaction mixture (R_{M}) wherein the trimerization is carried out in the presence of an apolar solvent. Useful apolar (inert) solvents are, for example, at least one solvent selected from the group consisting of benzene, cyclohexane, hexane, heptane, octane, decane, toluene and xylene. In accordance with the invention, the terms hexane, heptane, octane, decane and xylene encompass all isomers of these compounds. Preference is given to a reaction mixture (R_{M}) wherein the trimerization is carried out in the presence of toluene. Toluene is thus especially preferred among the apolar (inert) solvents.

The present invention thus also provides a process wherein the at least one apolar solvent is selected from the group consisting of benzene, cyclohexane, hexane, heptane, octane, decane and xylene.

It is particularly preferred when the reaction mixture (R_{M}) derives from the trimerization reaction of 1,3-butadiene described in WO 2009092683.

Particularly preferred reaction mixtures (R_{M}) thus comprise 15% to 70% by weight of cyclododecatriene, 10% to 80% by weight of at least one apolar solvent and 0.01% to 5% by weight of the active catalyst system (C) wherein the % by weight figures are in each case based on the total weight of the reaction mixture (R_{M}). What has been said in connection with cyclododecatriene, the apolar solvent and the active catalyst system, including preferences, applies correspondingly in connection with the reaction mixture (R_{M}) described hereinabove.

The present invention thus also provides a process wherein process step a) comprises adding to the reaction mixture (R_{M}) 0.1 to 20 g of gaseous ammonia per 1 kg of the reaction mixture (R_{M}).

The % by weight figures relating to the reaction mixture (R_{M}) sum to 100% by weight.

The present invention thus also provides a process wherein the active catalyst system (C) comprises at least one organoaluminum compound selected from the group consisting of Al₂(C₂H₅)₆. Al₂Cl₃(C₂H₅)₃ and AlCl(C₂H₅)₂ and at least one titanium compound selected from the group consisting of titanium tetrachloride and titanium acetylacetonate.

The active catalyst system (C) comprised in the reaction mixture (R_{M}) is in particular a catalyst system (C) obtainable from titanium tetrachloride and ethylaluminum sesquichloride (Al₂Cl₃(C₂H₅)₃) and water wherein 2 to 50 mol of ethylaluminum sesquichloride and 0.1 to 20 mol of water are employed per 1 mol of titanium tetrachloride.

### Process Step a)

Process step a) comprises contacting the reaction mixture (R_{M}) described hereinabove with gaseous ammonia. This gives a first mixture (M1) comprising cyclododecatriene and a first deactivated catalyst system (C1).

It is believed that the contacting with gaseous ammonia converts the organoaluminum compound comprised in the reaction mixture (R_{M}) into the amidoaluminum chlorides described in the introductory part of the present invention's description. This converts the active catalyst system (C) into the first deactivated catalyst system (C1). The first mixture (M1) thus comprises aluminum compounds having a vapor pressure of more than 1000 mbar at 400° C.

In accordance with the invention, the determination of the vapor pressure is effected according to the “OECD Guideline for the Testing of Chemicals; 104” of Jul. 27, 1995.

The present invention thus also provides a process wherein the first mixture (M1) comprises cyclododecatriene and a first deactivated catalyst system (C1) wherein the first mixture (M1) comprises aluminum compounds having a vapor pressure of more than 1000 mbar at 400° C.

It is believed that the aluminum compounds having a vapor pressure of more than 1000 mbar at 400° C. are the amidoaluminum chlorides described by way of introduction. The beliefs described hereinabove are not intended to limit the present invention.

Direct distillative removal of cyclododecatriene from the first mixture (M1) would co-vaporize the aluminum compounds having a vapor pressure of more than 1000 mbar at 400° C. and lead to deposits in the distillation apparatus. It is believed that the conditions of the distillative removal of cyclododecatriene from the first mixture (M1) described in the prior art processes generate polymeric aluminum compounds. It is believed that the vaporizable amidoaluminum chlorides undergo a condensation reaction with elimination of ammonia and/or hydrogen chloride to form polymeric compounds of the empirical formulae (AlN), and/or (AlClNH), or that they undergo hydrolysis to form insoluble compounds in further steps of the distillative work-up. These condensation products (deposits) are insoluble in organic solvents and can be removed from the distillation apparatus only with a great deal of mechanical effort.

The contacting of the reaction mixture (R_{M}) with gaseous ammonia may be carried out at temperatures in the range of from 20° C. to 140° C., preferably in the range of from 30° C. to 90° C., and at a pressure in the range of from 0.5 bar_{abs} to 50 bar_{abs}, preferably in the range between 1 bar_{abs} and 5 bar_{abs}.

The gaseous ammonia is preferably anhydrous. In the present case, “anhydrous” is understood to mean that the gaseous ammonia comprises less than 1% by weight, preferably less than 0.5% by weight, more preferably less than 0.1% by weight, of water in each case based on the total weight of the gaseous ammonia employed in process step a).

Process step a) generally comprises contacting the reaction mixture (R_{M}) with 5 to 20 g, preferably with 1 to 5 g, of gaseous ammonia in each case based on 1 kg of the reaction mixture (R_{M}). One preferred embodiment generally comprises adding to the reaction mixture (R_{M}) 0.5 to 5 g, preferably 1 to 3 g, of aqueous ammonia per 1 kg of reaction mixture (R_{M}).

The present invention thus also provides a process wherein process step a) comprises adding to the reaction mixture (R_{M}) 0.1 to 20 g of gaseous ammonia per 1 kg of the reaction mixture (R_{M}).

One preferred embodiment comprises adding to the reaction mixture (R_{M}) 0.1 to 50 mol, preferably 1 to 40 mol, of gaseous ammonia per 1 mol of the organoaluminum compound comprised in the reaction mixture (R_{M}).

Process step a) is preferably carried out as a continuous operation. Useful apparatuses for contacting the reaction mixture (R_{M}) with gaseous ammonia include stirred tanks, stirred-tank cascades and tubular reactors for example. The contacting of the reaction mixture (R_{M}) with gaseous ammonia is preferably carried out over a period of at least 0.1 hour, more preferably at least 0.3 hour, yet more preferably at least 0.5 hour and most preferably at least 1 hour. The contacting may be carried out over any desired length of time according to process step a). However, the period is generally no longer than 24 hours. Process step a) is thus preferably carried out over a period in the range of from 0.1 to 24 hours, more preferably over a period in the range of from 0.3 to 12 hours and most preferably over a period in the range of from 0.5 to 5 hours.

The present invention thus also provides a process wherein process step a) is carried out over a period of at least 0.1 hours.

The period thus describes the duration of process step a), i.e. the period from the initial contacting of the reaction mixture (R_{M}) with gaseous ammonia up until the contacting of the first mixture (M1) with water according to process step b).

The first mixture (M1) is formed of only one liquid phase during process step a).

The present invention thus also provides a process wherein the first mixture (M1) comprises only one liquid phase.

The formation of two liquid phases occurring in the processes described in the prior art is avoided using the process according to the invention. Process step a) is moreover accompanied by the formation of essentially no precipitate. In accordance with the invention, “essentially no precipitate” is understood to mean the precipitation of no more than 1% by weight, preferably no more than 0.5% by weight and more preferably no more than 0.25% by weight of solid in each case based on the total weight of the first mixture (M1). Thus, in one preferred embodiment, the first mixture (M1) may be supplied directly to process step b) without a phase separation step or a filtration step.

The present invention thus also provides a process wherein the first mixture (M1) comprises less than 1% by weight of solid based on the total weight of the first mixture (M1).

### Process Step b)

Process step b) comprises contacting the first mixture (M1) obtained in process step a) with water. This decomposes the aluminum compounds having a vapor pressure of more than 1000 mbar at 400° C. comprised in the first mixture (M1) to give the second deactivated catalyst system (C2). The second mixture (M2) thus comprises no aluminum compounds having a vapor pressure of more than 1000 mbar at 400° C.

The present invention thus also provides a process wherein the second mixture (M2) comprises cyclododecatriene and a second deactivated catalyst system (C2) wherein the second mixture (M2) comprises no aluminum compounds having a vapor pressure of more than 1000 mbar at 400° C.

It is believed that the addition of water converts the vaporizable amidoaluminum chlorides comprised in the first mixture (M1) into nonvaporizable oxidic compounds of aluminum.

Process step b) comprises contacting the first mixture (M1) obtained in process step a) with just sufficient water to completely dissolve the water in the second mixture (M2) obtained.

Process step b) preferably comprises adding only sufficient water for the second mixture (M2) to comprise only one liquid phase. This obviates the need for the phase separation step which is mandatory in the processes described in the prior art. One preferred embodiment of the present invention comprises supplying the second mixture (M2) to the distillative removal of cyclododecatriene from the second mixture (M2) according to process step c) without a prior phase separation step, i.e. without the separation of two liquid phases.

One preferred embodiment comprises adding water to the first mixture (M1) in an amount of 0.05 to 1.0 g, more preferably 0.05 to 0.5 g and most preferably 0.05 to 0.2 g per 1 kg of the first mixture (M1).

The present invention thus also provides a process wherein process step b) comprises adding to the first mixture (M1) 0.05 to 1.0 g of water per 1 kg of the first mixture (M1).

One preferred embodiment comprises adding to the first mixture (M1) from 0.1 to 10 mol, particularly preferably from 0.5 to 2 mol, of water per 1 mol of organoaluminum compound(s) originally comprised in the reaction mixture (R_{M}).

The contacting with water according to process step b) to obtain the second mixture (M2) is preferably carried out over a period of at least 0.5 hours, preferably at least 1 hour and more preferably at least 2 hours.

The contacting of the first mixture (M1) with water may be carried out over any desired length of time according to process step b). However, the period is generally no longer than 24 hours. Process step b) is preferably carried out over a period of 0.5 to 24 hours, more preferably 1 to 20 hours and most preferably 2 to 18 hours.

The present invention thus also provides a process wherein process step b) is carried out over a period of at least 0.5 hours.

The contacting according to process step b) is carried out at temperatures in the range of from 20° C. to 100° C. and at pressures in the range of from 0.5 bar_{abs} to 10 bar_{abs}. Process step b) is preferably carried out in a dwell time apparatus. This may be a continuously operated stirred tank or a continuously operated stirred tank cascade. However, preference is given to using a tube bundle reactor as the dwell time apparatus. Here, the contacting with water according to process step b) may be effected in the dwell time apparatus. However, it is preferred when the water is added to the first mixture (M1) prior to entry into the dwell time apparatus.

A second mixture (M2) is obtained during and/or on completion of process step b), said second mixture comprising a second deactivated catalyst system (C2) and comprising no aluminum compounds having a vapor pressure of more than 1000 mbar at 400° C. This is effective in preventing the formation of deposits during the distillative removal of cyclododecatriene from the second mixture (M2) in the distillation apparatus according to process step c).

The second mixture (M2) preferably comprises only one liquid phase,

The present invention thus also provides a process wherein the second mixture (M2) comprises only one liquid phase.

The second mixture (M2) obtained in process step b) is preferably supplied to process step c) without a liquid-liquid phase separation step being carried out. In accordance with the invention, “phase separation step” is understood to mean the separation of two liquid phases.

Moreover, process step b) is surprisingly accompanied by the formation of only little, if any, precipitate (solid). The second mixture (M2) preferably comprises less than 10% by weight of solid, more preferably less than 5% by weight and in particular less than 1% by weight of solid in each case based on the total weight of the second mixture (M2).

The present invention thus also provides a process wherein the second mixture (M2) comprises less than 10% by weight of solid based on the total weight of the second mixture (M2).

In one embodiment, the second mixture (M2) may be supplied to process step c) without being subjected to a filtration step. The second mixture (M2) is preferably subjected to a filtration step prior to process step c) in order to remove any solid comprised in the second mixture (M2).

### Process Step c)

Process step c) comprises distillatively removing the cyclododecatriene from the second mixture (M2).

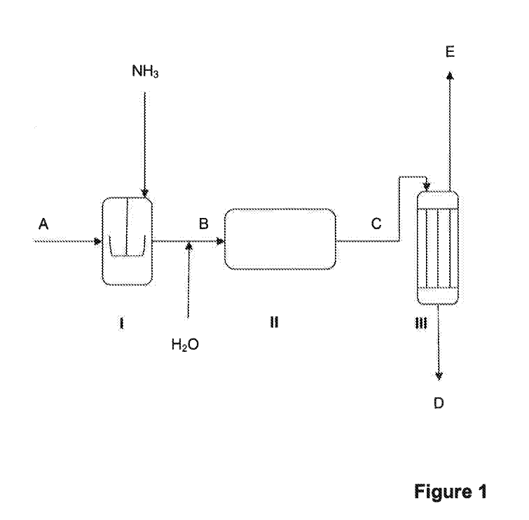

The distillative removal may be carried out at temperatures in the range of from 140° C. to 220° C. and at pressures in the range of from 10 mbar_{abs} to 1 bar_{abs}. Preference is given to a distillative removal of the type described in WO 2009092682 for example, Useful distillation apparatuses include distillation columns or evaporators for example, evaporators being preferred. Process step c) comprises overhead removal of the target product cyclododecatriene, any apolar solvents present, preferably toluene, and any additional low boilers. High polymers and the second deactivated catalyst system (C2) are removed in the bottoms from the distillation apparatus. The present invention is more particularly described with reference to FIG. 1.

The reference numerals in FIG. 1 are defined as follows:
A Stream comprising the reaction mixture (R_{M})
B Stream comprising the first mixture (M1)
C Stream comprising the second mixture (M2)
D Distillation apparatus bottoms
E Stream comprising the target product cyclododecatriene and any apolar solvent
I Apparatus for performing process step a)
II Dwell time vessel for performing process step b)
III Distillation apparatus for removing cyclododecatriene in accordance with process step c)

FIG. 1 depicts stream A comprising the reaction mixture (F_{M}) comprising cyclododecatriene and an active catalyst system (C) being supplied to a stirred tank I. Gaseous ammonia is supplied to the reaction mixture (R_{M}) in apparatus I via a feed line. This converts the reaction mixture (R_{M}) into the first mixture (M1). The first mixture (M1) comprises aluminum compounds having a vapor pressure of more than 1000 mbar at 400° C. It is believed that these are the amidoaluminum complexes described hereinabove. Stream B comprising the first mixture (M1) is supplied to dwell time vessel II from apparatus I. Water is added to stream B prior to entry into the dwell time vessel II. The first mixture (M1) is converted into the second mixture (M2) in the dwell time vessel II, said second mixture (M2) comprising no aluminum compounds having a vapor pressure of more than 1000 mbar at 400° C. The residence time in the time reactor II is at least 1 hour. The second mixture (M2) is supplied to the evaporator III via stream C. It is preferable when there is a filter apparatus interposed between the dwell time reactor II and the evaporator III in order to remove any generated solid prior to entry into the evaporator III. Stream E comprising cyclododecatriene, any apolar solvents present and any further volatile by-products is removed from the evaporator III overhead. The cyclododecatriene may be further purified if desired. Stream D comprising the second deactivated catalyst system (C2) and also by-produced high polymers and high-boilers is removed from the bottom of evaporator III.

The present invention is more particularly described using the examples and comparative examples which follow but is not limited thereto.

### COMPARATIVE EXAMPLE C1

4.6 mg of titanium tetrachloride (1% strength solution in benzene) and 152 mg of aluminum sesquichloride (5.48% strength solution in toluene) are dissolved in 12.25 g of benzene under inert conditions. This mixture is transferred into a glass autoclave. 19.2 ml of 1,3-butadiene (corresponding to about 12 g) are subsequently added to the glass autoclave at 70° C. over a period of 1 hour. This establishes an overpressure of 0.4 bar.

On completion of the reaction the reaction mixture (R_{M}) thus obtained is removed from the autoclave and divided in two. To the first half is added 0.15 ml of a 25% strength aqueous ammonia solution. To the second half of the reaction mixture (R_{M}) are added 0.15 ml of a 25% strength aqueous ammonia solution and then 0.25 g of water.

In both cases a white precipitate is brought down from the reaction mixture (R_{M}) and two liquid phases are formed. The distillative removal of cyclododecatriene must therefore be preceded by a liquid-liquid phase separation step and a filtration step to remove the precipitated solid.

Comparative example C1 describes a process as described in DE 1768067. Here, comparative example C1 replicates working examples 1a and 1 b of DE 1 768 067.

### COMPARATIVE EXAMPLE C2

To a 1 liter glass reactor are initially charged 504 g of a reaction mixture (R_{M}) comprising 57.7% by weight of cyclododecatriene, 34.0% by weight of toluene, 0.04% by weight of water, 1.8% by weight of aluminum sesquichloride and 0.14% by weight of titanium tetrachloride. The remainder of the reaction mixture (R_{M}) is composed of high polymers, high boilers and other by-products. 5.2 g of gaseous ammonia are then added to the reaction mixture (R_{M}) at 60° C. and the mixture obtained is stirred for 4 hours. This generates a slightly cloudy solution. However, no precipitate is brought down even after cooling. The mixture thus obtained corresponds to the first mixture (M1) of the process according to the invention.

This first mixture (M1) is subsequently subjected to distillative work-up. Here, cyclododecatriene and toluene are removed overhead. White deposits form in the distillation apparatus after an operating time of 7 days. These white deposits are insoluble in organic solvents and can be removed only with a great deal of mechanical effort.

### INVENTIVE EXAMPLE I1

504 g of the reaction mixture (R_{M}) described hereinabove in comparative example C2 are introduced into a Mettler-Toledo RC1e reaction calorimeter provided with a SV01 glass reactor (1 liter). 5.2 g of gaseous ammonia are then added to this reaction mixture (R_{M}) at 60° C. and the mixture thus obtained is stirred for 4 hours. The calorimeter measures the heat evolved here which signals the formation of the first deactivated catalyst system (C1). The data regarding heat evolved demonstrate that the formation of the first deactivated catalyst system (C1) is complete after 100 minutes. The mixture thus obtained corresponds to the first mixture (M1) of the process according to the invention.

1 g of water is then added to this first mixture (M1) at 60° C. The heat evolved by the reaction is likewise monitored by calorimetry. The calorimetric data show that formation of the second mixture (M2) is complete after 133 minutes. The second mixture (M2) comprises only a single liquid phase here, thereby rendering a liquid-liquid phase separation unnecessary. The second mixture (M2) moreover exhibits only a minimal amount of precipitate which may be filtered off if desired. The mixture thus obtained corresponds to the second mixture (M2) of the process according to the invention.

The second mixture (M2) is subsequently supplied to a distillation apparatus in order to remove cyclododecatriene and toluene overhead.

Visual inspection of the distillation apparatus reveals no white deposits even after a distillation apparatus operating time of 7 days.

The inventive example I1 demonstrates that a liquid-liquid phase separation step is unnecessary with the process according to the invention. The process according to the invention is moreover accompanied by the precipitation of only minimal amounts of solid. The process according to the invention is also effective at preventing the formation of deposits in the distillation apparatus. This facilitates a distinctly simplified and thus more cost-effective procedure and an uninterrupted continuous operation for the work-up of a reaction mixture (R_{M}) comprising cyclododecatriene.

1-14. (canceled)

15. A process for the work-up of a reaction mixture (R_{M}) comprising cyclododecatriene and an active catalyst system (C) comprising an organoaluminum compound, said process comprising the steps of:
a) contacting the reaction mixture (R_{M}) with gaseous ammonia to obtain a first mixture (M1);
b) contacting the first mixture (M1) with water to obtain a second mixture (M2); and
c) distillatively removing cyclododecatriene from the second mixture (M2).

16. The process according to claim 15, wherein the first mixture (M1) comprises cyclododecatriene and a first deactivated catalyst system (C1), and wherein the first mixture (M1) comprises aluminum compounds having a vapor pressure of more than 1000 mbar at 400° C.

17. The process according to claim 15, wherein the second mixture (M2) comprises cyclododecatriene and a second deactivated catalyst system (C2), and wherein the second mixture (M2) comprises no aluminum compounds having a vapor pressure of more than 1000 mbar at 400° C.

18. The process according to claim 15, wherein the second mixture (M2) comprises only one liquid phase.

19. The process according to claim 15, wherein process step a) comprises adding to the reaction mixture (R_{M}) 0.1 to 20 g of gaseous ammonia per 1 kg of the reaction mixture (R_{M}).

20. The process according to claim 15, wherein process step b) comprises adding to the first mixture (M1) 0.05 to 1.0 g of water per 1 kg of the first mixture (M1).

21. The process according to claim 15, wherein the reaction mixture (R_{M}) comprises:
a) 10% to 70% by weight of cyclododecatriene;
b) 10% to 80% by weight of at least one apolar solvent; and
c) 0.5% to 5% by weight of the active catalyst system (C),
wherein the % by weight values are in each case based on the total weight of the reaction mixture (R_{M}).

22. The process according to claim 15, wherein the active catalyst system (C) comprises at least one organoaluminum compound selected from the group consisting of Al₂(C₂H₅)₆, Al₂Cl₃(C₂H₅)₃ and AlCl(C₂H₅)₂ and at least one titanium compound selected from the group consisting of titanium tetrachloride and titanium acetylacetonate.

23. The process according to claim 21, wherein the at least one apolar solvent is selected from the group consisting of benzene, cyclohexane, hexane, heptane, octane, decane and xylene.

24. The process according to claim 15, wherein process step a) is carried out over a period of at least 0.1 hours.

25. The process according to claim 15, wherein process step b) is carried out over a period of at least 0.5 hours.

26. The process according to claim 15, wherein the first mixture (M1) comprises only one liquid phase.

27. The process according to claim 15, wherein the first mixture (M1) comprises less than 1% by weight of solid based on the total weight of the first mixture (M1).

28. The process according to claim 15, wherein the second mixture (M2) comprises less than 10% by weight of solid based on the total weight of the second mixture (M2).
